(19) United States
(12) Patent Application Publication
(10) Pub. No.: US 2011/0223057 A1
(43) Pub. Date: Sep. 15, 2011

(54) AIR DECONTAMINATION EQUIPMENT

(76) Inventors: Renato Della Valle, London (GB); Carlo Alberto Bignozzi, London (GB)

(21) Appl. No.: 13/057,950

(22) PCT Filed: Jul. 21, 2009
(86) PCT No.: PCT/IT2009/000323

§ 371 Date: May 4, 2011

(30) Foreign Application Priority Data
Aug. 8, 2008 (IT) MI2008A001502

(51) Int. Cl. A61L 9/18

(52) U.S. Cl. 422/4; 422/122

(57) ABSTRACT

The present invention relates to an air decontamination equipment, from both odours or pollutants, and bacterial or viral loads. More particularly, the present invention relates to a decontaminating equipment (1) for the treatment of air, comprising a shell (2) which is divided in a first and a second compartments (3, 4), which are arranged in a contiguous position in any sequence order, in the second of said compartments (3, 4) suction means (6) being arranged, in which one of said first and second compartments (3, 4) is for the antibacterial/antiviral treatment of air, and one of said first and second compartments (3, 4) is for the photocatalytic treatment of air, and comprises UV illumination means (9), said first and second compartments (3, 4) comprising a material with antibacterial and antiviral activity and a material with photocatalytic activity, respectively.

The present invention relates to an air decontamination equipment, from both odours or pollutants, and bacterial or viral loads.

### STATE OF THE ART

In domestic, industrial, hospital environments, in offices, shops, or in private and public spaces generally, air purification means are used which have different configurations in order to take into account particular needs.

Furthermore, in such environments there is very often the need to purify and/or filter air, for example, to reduce the smoke that is present moreover in the public spaces, or the particulate that is generated, for example, by an industrial processing, or odours produced by a kitchen, or the pollutants that are present in the air, such as NOx, SOx, CO, organic vapours, C₆H₆, etc., in order to make the permanence in such environments more pleasant and salubrious.

In public, above all hospital, environments, there is further the need to eliminate possible viruses or bacteria which are present in the air, in order to maintain high hygienic conditions within such environments, possibly substantially sterile conditions.

In the railway, public transportation, naval, and aeroplane field, the air recycle and filtration are necessary to allow comfort and well-being to the passengers.

In the domestic field, in kitchen hoods, filters of different types and materials are used, to reduce the odours generated by the food itself. Said filters have very short saturation times compared to those described in the invention, and very high load losses. Furthermore, these filters are full with bacteria after a few days of use. Again in the domestic field, filters would be desirable in refrigerators, which would be able to reduce odours, for food preservation and reduction of the bacteria deriving from the decomposition of the food itself.

The functions indicated above are performed by the known purification means, such as fans, air cleaners, air treatment plants, air conditioners, kitchen hoods, ventilation or conditioning systems of cars, trucks, motor buses, aeroplanes, trains, ships, which use filters that do not eliminate bacteria, rather allowing the proliferation thereof, and do not eliminate, if not by adsorption (activated carbons), the urban pollutants (temporarily), such as NOx, SOx, CO, C₆H₆, CO₂, O₃, etc. Furthermore, they do not eliminate the odours, and allow the proliferation of molds.

The antibacterial function of some metal ions, also referred to as oligodynamic effect, is known.

Metal ions having the highest antibacterial activity are, in a decreasing effect order, ions of the following metals:

Hg>Ag>Cu>Zn>Fe>Pb>Bi

The inclusion of such metals, particularly of silver ions, in plastic materials, ceramics, and fibers, or carbon-based materials, allows reducing or eliminating the growth of bacterial colonies. This effect is particularly relevant, given the compatibility of Ag⁺ with the human body and the growing antibiotic resistance of many bacteria. The use of silver-containing materials can thus perform the preventive function of limiting or avoiding the bacterial proliferation.

At the current state of the art, the production of nanocrystalline materials with high surface development is further known, which are based on metal oxides (MOₓ), such as titanium dioxide, zinc oxide, tin dioxide, zirconium dioxide, and colloidal silica, which can be stably deposited and adhered to different substrates. Such materials, above all if irradiated with UV light, are capable of performing a photocatalytic effect on pollutants and odours, thus causing the elimination thereof, or at least a reduction thereof. The above-described nanocrystalline materials also perform an antibacterial or antiviral activity, although only after contact times of some hours.

A further evolution of such nanocrystalline materials has lead to the development of innovative antibacterial and antiviral nanomaterials based on metal or metalloid oxides, such as, for example, TiO₂, ZrO₂, SnO₂, ZnO, and SiO₂, functionalized with molecular species, of an organic or organometallic nature, which are capable of simultaneously binding both the oxide and ions of transition metals, such as, for example, Ag⁺ or Cu²⁺ (Patent Publication WO 2007/122651 by the same Applicant).

### SUMMARY OF THE INVENTION

It has been now found that it is possible to decontaminate air from both the bacterial and/or viral load contained therein, and chemical pollutants and/or malodours in short times (a few minutes) and with maximum efficiency.

Therefore, the object of the present invention is an air decontamination apparatus, consisting of a first section which is treated with a nanocrystalline material of formula (I) defined herein below, having antibacterial and antiviral activity, and a second section with photocatalytic activity, comprising a photocatalytic nanocrystalline material as defined herein below. The arrangement along the airflow being treated of the antibacterial section and the photocatalytic section can also be inverted, therefore putting the photocatalytic section before the antibacterial/antiviral one. Therefore, in the present description, the term “first section” or “second section” will not necessarily mean a particular spatial arrangement.

The nanocrystalline materials with antibacterial and/or antiviral activity of said first section of the apparatus of the invention have formula (I):

AOₓ-(L-Meⁿ⁺)ᵢ (I)

where

AOₓ represents the metal or metalloid oxide, with x=1 or 2;

Meⁿ⁺ is a metal ion selected from Ag⁺ or Cu⁺⁺,

L is a bifunctional molecule, organic or organometallic, capable of concomitantly binding both the metal or metalloid oxide and the metal ion Meⁿ⁺, and

i represents the number of L-Meⁿ⁺ groups linked to an AOₓ nanoparticle, where i ranges between 10² and 10⁶.

The AOₓ metal or metalloid oxides which can be used within the scope of the present invention are, for example: colloidal silica, titanium dioxide, zirconium dioxide, tin dioxide, and zinc oxide. They are insulating or semiconductor materials which are capable of adhering as such, or by the application of a suitable primer, to a large number of materials including: wood, plastic, glass, metals, ceramics, cement, and inner and outer surfaces of buildings, and can be produced with nanoparticles dimensions in the range of the nanometers. These nanomaterials are capable of adsorbing, by electrostatic or chemical interaction, for example, through ester-type linkages, molecules which are provided with suitable functionalities, such as, for example, the carboxyl (—COOH), phosphoric (—PO₃H₂), or boronic (—B(OH)₂) groups, with which the bifunctional molecules L can be provided. Given the lower dimensions of the ligands L and of the metal ions Meⁿ⁺, for example, silver or copper, which can be placed in the range of the picometers, it results that each metal oxide nanoparticle can be homogeneously coated with metal ions such as Ag⁺ or Cu²⁺, as schematically set forth by way of illustrative example in FIG. 2.

It results that these nanomaterials, being composed of positively charged nanoparticles, can originate stable and transparent suspensions in both aqueous solvents and in organic polar solvents.

Another relevant aspect relates to the possibility to mix the suspensions of the nanomaterials of the invention with cationic surfactants, such as alkyl ammonium salts or with chlorhexidine digluconate. The bactericidal activity of the nanomaterial suspensions of the invention can be thus enhanced by the presence of the cationic surfactant.

In fact, experimental proofs indicate that the cationic surfactants such as benzalkonium chloride can originate an adsorption to the surface of titanium dioxide-based nanomaterials. This provides the further advantage of reducing the volatility of the alkyl ammonium salt once this has been applied to a surface.

The photocatalytic section of the air decontamination apparatus is treated with Titanium dioxide in the Anatase crystal form. The photocatalytic properties of titanium dioxide in the Anatase allotropic form have been studied by many research groups with the aim of developing methods and apparatus for water and air purification. Examples of these works are described in the literature references (Ollis, D.; F. Pelizetti E.; Serpone N. Environ Sci. Technol. 1991, 25, 1523; Uccida, H.; Itoh, S.; Yoneyama, H. Chem. Lett. 1993, 1995; Heller, A. Acc. Chem. Res. 1995, 28, 503; Sitkiewitz, S; Heller, A. New J. Chem 1996, 20 233. These properties are related to the strong oxidative ability of the material undergoing irradiation with UV light. The efficacy of titanium dioxide-coated materials in deodorizing the surrounding environment and the self-cleaning properties thereof have been widely investigated; see, for example, the works (Watanabe, T; Kitamura, A.; Kojima, E.; Nakayama, C; Hashimoto, K; Fujishima, A; In Photocatalytic Purification and Treatment of Water and Air; 011 is D. E., Al-Ekabi, H; Eds; Elsevier: New York, 1993, 747; Matsubara, H,; Takada, M; Koyama, S.; Hashimoto, K.; Fujishima, A. Chem. Lett. 1995, 767; Negishi, N.; Iyoda, T; Hashimoto, K.; Fujishima, A. Chem. Lett. 1995, 841; Sunada, K.; Kikuki, Y; Hashimoto, K.; Fujishima, A. Environ Sci Technol, 1998, 32, 726; Ichinose, H.; Terasaki, M.; Katsuki, H. J. Of Ceramic Soc. of Japan, 1996, 104, 715).

The microbicidal action of titanium dioxide irradiated with UV light has been also investigated and verified before (SUSPENSIONS OF TITANIUM DIOXIDE AND METHOD FOR OBTAINING THEM″, PCT publication No. WO2006/136931).

### BRIEF DESCRIPTION OF THE DRAWINGS

FIG. 1 shows a block chart of the apparatus of the invention;

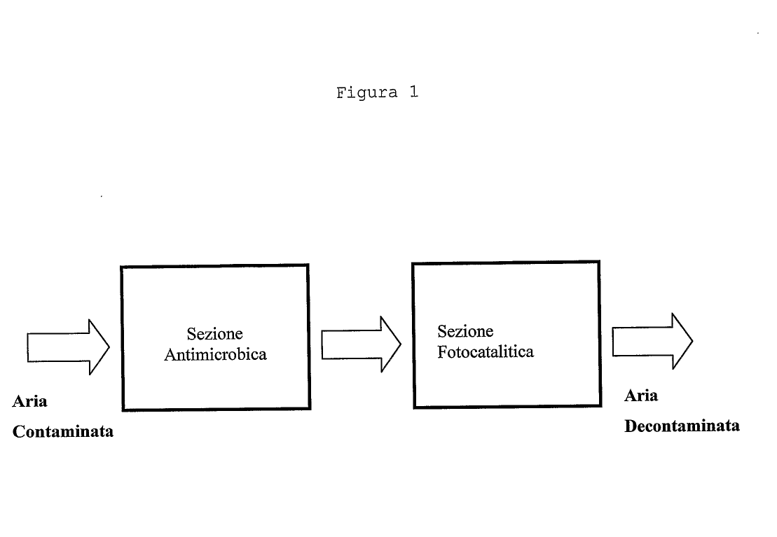

FIG. 2 shows a schematic view of the structure of a nanoparticle with antibacterial activity according to the invention;

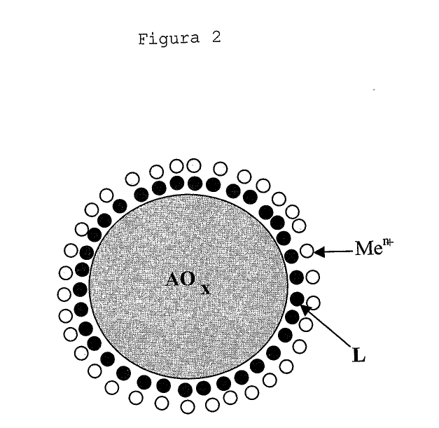

FIG. 3 shows a schematic view of a possible decontamination equipment according to the invention;

FIG. 4 shows the decay of a NOx mixture with an initial concentration equal to 0.65 ppm, under irradiation conditions of the photocatalytic filter (Light) and in the absence of irradiation (Darkness).

### DETAILED DESCRIPTION OF THE INVENTION

The present invention relates to an air decontamination apparatus, consisting of a first section treated with a nanocrystalline material of formula (I), having antibacterial and antiviral activity, and a second section with photocatalytic activity, comprising a photocatalytic nanocrystalline material.

The antibacterial/antiviral nanocrystalline compounds are comprised in the formula (I):

AOₓ-(L-Meⁿ⁺)ᵢ (I)

where

AOₓ represents the metal or metalloid oxide, with x=1 or 2;

Meⁿ⁺ is a metal ion with antibacterial activity, selected from Ag⁺ or Cu⁺⁺;

L is a bifunctional molecule, organic or organometallic, capable of concomitantly binding both the metal or metalloid oxide and the metal ion Meⁿ⁺; and

i represents the number of L-Meⁿ⁺ groups linked to an AOₓ nanoparticle, in which i ranges between 10² and 10⁶.

The value of the parameter i will depend on several factors, such as the AOₓ nanoparticle size, the nature of the ligand L, and the method, which is used for the preparation thereof. Within the scope of the present invention, i will correspond to the number of ligands L that the nanoparticle AOₓ is capable of binding when said nanoparticle is contacted with a solution of ligand L for a period of time ranging between 10 minutes and 72 hours, preferably between 3 and 24 hours.

The nanomaterials of the present invention have a particle size ranging between 10 and 400 nm. Titanium dioxide nanoparticles with dimensions below 20 nm generally result in transparent suspensions allowing a wider range of applications. SiO₂-based nanoparticles result in transparent suspensions in water, even if the dimensions thereof are higher (200-400 nm), since they have a refractive index which is similar to that of water.

The AOₓ metal or metalloid oxides which can be used within the scope of the present invention are, for example: colloidal silica, titanium dioxide, zirconium dioxide, tin dioxide, and zinc oxide.

Bifunctional Ligands L Based on Transition Metals Complexes

The transition metals complexes that are useful for this use must contain organic ligands, coordinated at the metallic centre, with boronic, B(OH)₂, phosphonic, PO₃H₂, or carboxyl, COOH, functionalities. Such functionalities have as their aim to bind the complex to the AOₓ nanocrystalline substrate. The other groups, coordinated at the metallic centre, must be capable of binding metal ions with antibacterial activity. Examples of these groups include ligands of the Cl⁻, Br⁻, I⁻, CNS⁻, NH₂, CN⁻, and NCS⁻ type.

The metallorganic complexes L according to the invention preferably comprise organic ligands of the dipyridyl and/or terpyridyl type, coordinated at a metallic centre (M), functionalized with carboxyl COOH, boronic B(OH)₂, or phosphonic PO₃H₂ groups capable of bonding to nanomaterials comprised of AOₓ; and Cl⁻, Br⁻, I⁻, CNS⁻, NH₂, CN⁻ or NCS⁻ groups which are coordinated at said metallic centre (M), capable of bonding to Ag⁺ or Cu²⁺ ions. Preferably, said dipyridylic or terpyridylic groups will be substituted with carboxyl groups, more preferably in the para position with respect to the pyridine nitrogen. In the case where more than one dipyridyl or terpyridyl group is present in said organometallic complex L, optionally one of said groups may be unsubstituted.

Concerning the metal ions (M) present in L, having coordinations of the octahedral type, or having other types of coordination corresponding to the tetrahedral, square-planar, bipyramidal trigonal, squared base pyramidal geometries, all the metals of the first, second, and third row of transition metals in the periodic table of the elements which can give rise to stable bifunctional molecules of the described type can be included.

More preferably, such metallorganic complexes L will have a coordination of the octahedral type. The transition metals coordinated by said complexes will be preferably selected from Cr, Mn, Fe, Co, Ni, Cu, Zn, Ru, Rh, Pd, Re, Os, Ir, Pt.

The metallorganic complexes L of the invention may also have a negative charge, and will therefore form salts with cations, preferably organic cations such as tetraalkylammonium cations. Such cations allow the solubilisation of these species in organic solvents, which promote the adsorption process on the nanomaterials based on metal or metalloid oxides.

Thus, such molecules can serve as bifunctional ligands capable of giving rise to an evenly adsorbed layer on the AOₓ nanoparticles, and at the same time of binding metal ions with antibacterial activity.

Examples of such complexes which have octahedral coordination are set forth herein below.

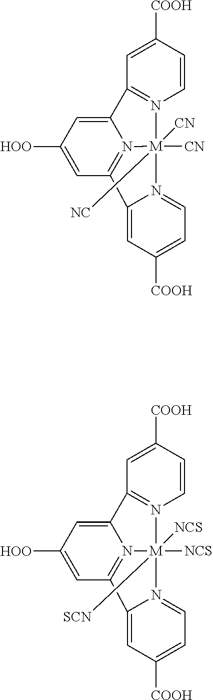

### [(H₃ Tcterpy)M(CN)₃]TBA [(H₃Tcterpy)M(NCS)₃]TBA

TBA=tetrabutylammonium cation
H₃Tcterpy=4,4′,4″-tricarboxy terpyridyl

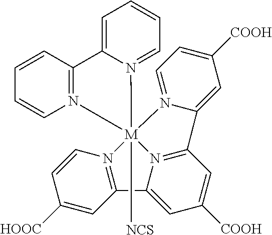

bpy=2,2′ dipyridyl
   [M(H₃tcterpy)(bpy)NCS]TBA

The TBA group can be replaced by another alkylammonium cation, which allows the solubilisation of the complex in organic solvents.

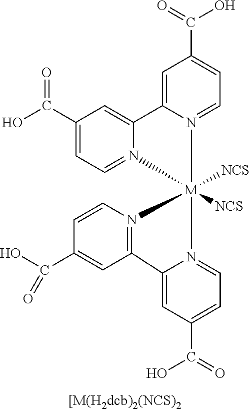

H₂dcb=4,4′-dicarboxy-2,2′ dipyridyl acid Bifunctional Ligands L Based on Organic Compounds

The bifunctional ligands L of an organic type that are usable in the context of the present invention include molecular species containing groups which can give rise to an interaction with AOₓ nanoparticles, and other functionalities which are capable of bonding ions with antibacterial activity. Examples of these molecular species include organic molecules containing carboxyl COOH, phosphonic PO₃H₂, and boronic B(OH)₂ functionalities which are capable of promoting the adsorption onto the surface of the AOₓ oxide; and N, NH₂, CN, NCS, or SH groups which are capable of bonding metal ions with antibacterial activity such as Ag⁺ or Cu²⁺ ions.

Such organic ligands will be preferably selected from:
nitrogen-containing heterocycle with 6-18 members, preferably selected from pyridine, dipyridyl, or terpyridyl, substituted with one or more substituents selected from carboxyl COOH, boronic group B(OH)₂, phosphonic group PO₃H₂, mercaptan SH, hydroxyl OH;
C6-C18 aryl, preferably selected from phenyl, naphthyl, diphenyl, substituted with one or more substituents selected from carboxyl COOH, boronic group B(OH)₂, phosphonic group PO₃H₂, mercaptan SH, hydroxyl OH;
C2-C18mono- or di-carboxylic acid, substituted with one or more mercaptan SH and/or hydroxyl OH groups.

Examples of these organic bifunctional ligands more preferably include pyridine, dipyridyl, or terpyridyl functionalized with carboxyl, boronic or phosphonic groups; mercaptosuccinic acid, mercaptoundecanoic acid, mercaptophenol, mercaptonicotinic acid, 5-carboxypentanethiol, mercaptobutyric acid, 4-mercaptophenyl-boronic acid, and 4-mercaptophenyl-phosphonic acid.

The suspensions of the nanomaterials of formula (I) can be mixed with cationic surfactants, as the alkyl ammonium salts, or with chlorhexidine digluconate. The bactericidal activity of the nanomaterial suspensions of the invention can be thus enhanced by the presence of the cationic surfactant.

The preparation of said nanocrystalline materials is known, and it can be carried out in accordance with the methods described in the patent publication WO 2007/122651 of the same Applicant. Such materials are further commercially available under the trade name Bactercline Multiuso of the company NM TECH SRL (medical/surgical device No. 19258).

The application of the nanocrystalline materials of formula (I) to the filters of the antibacterial section of the inventive equipment can be obtained from a solution thereof by means of spraying, painting, or dip-coating.

Nanocrystalline Materials with Photocatalytic Activity

The photocatalytic section of the apparatus according to the invention comprises, as already stated, a nanocrystalline material with photocatalytic activity.

Said material, hereinafter generally referred to as “photocatalytic material”, comprises a titanium dioxide layer, preferably in the form of anatase and/or modified peroxytitanic acid.

Preferably, said photocatalytic material comprises two or more titanium dioxide layers, preferably in the form of rutile, sandwiched between the treated surface and said first photocatalytic titanium dioxide layer.

In another version, said photocatalytic material comprises one or more further photocatalytic titanium dioxide layers in the form of peroxytitanic acid or other compounds with a strong adhesion power and non-oxidable, sandwiched between the treated surface and said first photocatalytic titanium dioxide layer.

In another version, said photocatalytic material further comprises titanium dioxide in the form of anatase and/or stabilizing surfactants.

In a further embodiment of the invention, said photocatalytic material further comprises at least one component selected from sodium hydroxide (NaOH), and silica (SiO₂).

The photocatalytic material according to the invention can be prepared and applied to the surface to be treated according to methods that are well known to those skilled in the art, such as those described in the patent publication WO 2007/026387 in the name of the present Applicant.

Filtering Material

The filtering material that can be used in the filters of the equipment of the present invention can be of different type.

In a first embodiment, the filtering material is made of ceramic material, preferably cordierite, composed as follows:

Cordierite ceramic filters having a squared shape or other, reticular, shape, having chemical composition (Fe,Mg)₂Al₄Si₅O₁₈.nH₂O, with 90% minimum content, besides to Mullite Al₆Si₂O₁₃, Aluminium oxide Al₂O₃, Spinel MgAl₂O₄, being the rest 10% material having a porosity ranging between 32% and 36%, and pore diameter of 3±1.5 μm, usable up to 1,380° C., having cells per square inch equal to 16CSI, 25CSI, 50CSI, 64CSI, 100CSI, 200CSI, 300CSI, 400CSI, 600CSI, with depth from 0.3 mm to 3,000 mm, or mixed.

In a second embodiment, the filtering material is made of polymer fibre, preferably in synthetic fibre of foamed polyester, impregnated with activated carbons, and consisting of:

Filter entirely composed of synthetic polyester fibre, also foamed, impregnated with activated carbon, mass per surface unit from about 10 g/m² to about 900 g/m², through speed of the filtering material from about 0.05 m/s to about 2.0 m/s. The filter has a nominal flow rate from about 0.100 m³/s to about 900 m³/s, and a load loss at 100% of the nominal flow rate from about 1 Pa to about 250 Pa, for those classified according to the EN 779 standard from G1 to G4, complying with the Eurovent standard from EU1 to EU4, and with a load loss at 100% of the nominal flow rate from about 1 Pa to about 450 Pa, for those classified according to the EN 779 standard from F5 to F9, complying with the Eurovent standard from EU5 to EU9, having a minimum absorption efficacy of about 75% for benzene (C₆H₆) on a concentration of 160000 μg/Nmc to a maximum absorption efficacy of about 97% on a concentration of 150 μg/Nmc. Alternatively, said filters are manufactured by means of another polymer fibre, of the type of polyester, thermoset polyester, polyurethane, also foamed polyurethane, cloth, also rotative and/or in the form of cups and/or paper, preferably also impregnated with activated carbons, or entirely filled with activated carbon, or mixed, or impregnated with Zeolite in pellets or in another form.

In a third embodiment, said filtering material is made of glass fibre (absolute filters Hepa and Ulpa with high and very high efficiency, respectively, classified as Hepa according to the EN 1822 standard from H10 to H14, complying with the Eurovent standard from EU10 to EU14, and classified as Ulpa according to the EN 1822 standard from U15 to U17, corresponding to the Eurovent standard from EU15 to EU17, which can have the filtering septum made of paper of glass micro fibres in small plies or deep plies, also with corrugated aluminium separators, with efficiency on particles from about 1.0 μm to 0.01 μm, or mixed).

In a fourth embodiment, the filtering material is made of plastic, also polypropylene (PP), modified polyphenyleneoxide (PPO), polycarbonate (PC), or polystyrene (PS), or sinterised foamed polystyrene (EPS) composed of a reduced-weight closed-cell rigid foamed material, or mixed. Generally, EPS has a volumetric mass ranging between 10 and 40 kg/mc, therefore it is composed of 98% by volume in average of air and only of 2% of pure hydrocarbon structural material.

In a fifth embodiment, the filtering material is supported on metallic supports, also in aluminium, both in the form of a mesh and sheet, in steel both in the form of a mesh (also inox) and sheet, or mixed.

Decontaminating Equipment

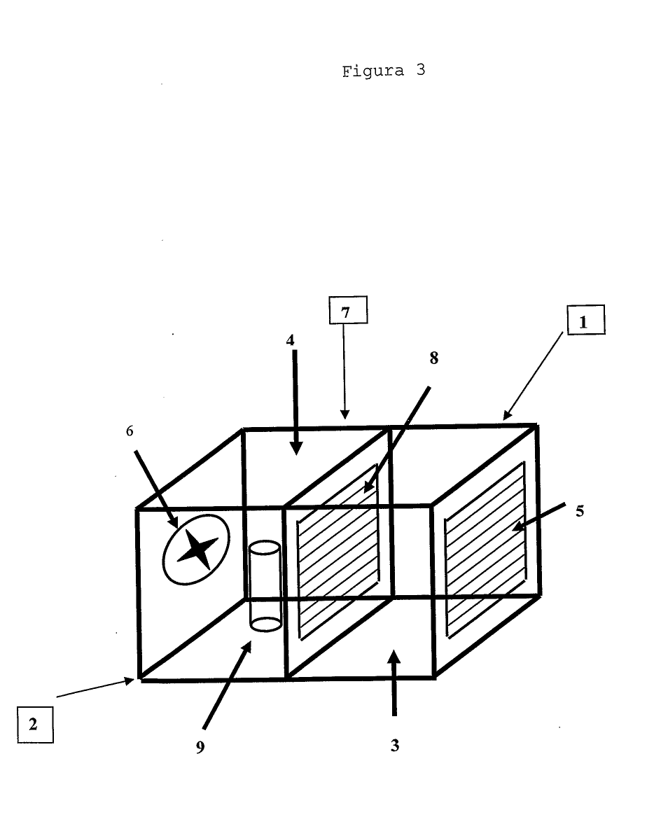

With reference to FIG. 3, that schematically shows a possible configuration of the equipment of the invention, the decontaminating equipment, generally indicated with the numeral 1, comprises a shell 2 which is divided into two compartments 3, 4 which are arranged in a contiguous position, a first compartment 3 for the antibacterial/antiviral treatment of air, and a second compartment 4 for the photocatalytic treatment of the air treated in said first compartment 3.

A first outer wall of the shell 2 confining with said first compartment 3 comprises a first filtering means 5 comprising a nanocrystalline material with antibacterial/antiviral activity of formula (I) as defined above.

A second outer wall of the shell 2, confining with said second compartment 4, comprises an opening communicating with the exterior of said compartment 4, and to which suction means 6 are associated.

Said first 3 and said second 4 compartments are separated by an inner wall 7 comprising second filtering means 8, to which a photocatalytic material as previously defined is associated.

In a preferred embodiment, also the inner surface of one or more walls of the compartment 4 is coated with said photocatalytic material.

A UV light source 9 is positioned within said second compartment 4, which serves to activate the photocatalytic material, allowing it to perform the decontaminating effect thereof against pollutants and/or odours.

The filtering means 5, 8 are made of a filtering material, for example, as defined above.

The shell 2 can be made of several materials, such as plastic or metals (aluminium or stainless steel).

The arrangement of the two compartments 3, 4 can also be inverted, to let air to pass first through the photocatalytic compartment, then through the antibacterial/antiviral compartment.

Experimental Section

With the aim of assessing the decontaminating ability of the decontaminating equipment of the invention against aero-dispersed microbial loads, an apparatus as described above has been manufactured, having dimensions of 20×15×15 cm, which is equipped with a suction fan and two filtering zones, where filters of different material could be inserted. A UV lamp which was present in the photocatalytic section allowed the irradiation of titanium dioxide deposited on the walls and the filter. The prototype was tested with filters being composed of glass wool or polyester. The filtering systems were inserted in frames having side dimensions of 14×14 cm, and a thickness equal to 0.5 cm. The used filters have been treated with titanium dioxide-based products in the main crystal form of Anatase, or with the Bactercline Multiuso antimicrobial product.

The forced ventilation system allows the monodirectional passage of air. The experiments of decontaminating air that is artificially polluted by microbial species or chemical pollutants, such as the nitrogen oxides, have been carried out in a Plexiglas chamber, called “Smog Chamber”, having a volume of 160 L. The Smog Chamber was divided into two compartments, and the decontaminating equipment 3 was inserted therebetween. In this manner, it has been possible to contaminate a compartment of the Smog Chamber and to analyse the decay of the concentration of microbial species or nitrogen oxides in the compartment downstream the decontaminating equipment. The contamination with microbial species of the Escherichia Coli type has been performed by vaporizing suspensions of micro-organisms with a known titre in the Smog Chamber.

### Assay System

### Micro-Organisms

The following test strain has been used:

Escherichia coli ATCC 10536

### Strain Collection

The bacteria, E. coli, come from the Dipartimento di Medicina Sperimentale e Diagnostica, Sezione di Microbiologia, of the University di Ferrara, and have been purchased from the company VWR International Srl. The bacterial strains have been kept frozen in culture broth and 50% glycerol (v/v); before use, they have been transplanted on TSA slant and preserved in a refrigerator at 4° C.±2° C.

| Culture media: | Tryptone Soya Agar (TSA) | |
|---|---|---|
| Diluent: | | |
| Tryptone, | | |
| Casein pancreatic digestion | 1.0 g | OXOID |
| NaCl | 8.5 g | MERCK |
| Distilled water, | q.s. 1000 ml | |

| | |
|---|---|
| Equipment used | |
| Oven for dry sterilization | KW |
| Vapour autoclave | COLUSSI |
| Thermostat | MEMMERT |
| Vortex stirrer | VELP |
| Chronometer | ARBORE |
| Micropipettes | GILSON |
| New Triflux 400 nebulizer | NUCLEOFARMA |

### Assessment of the Mortality of the Micro-Organisms Hold by the Filters

### Description of the Experimental Apparatus

The trials were carried out within a sealed Plexiglas chamber, with a volume of 160 L, referred to as a “Smog Chamber”.

The Smog Chamber is divided into two compartments by means of a plastic material septum, into which the decontaminating equipment of the invention is introduced.

On the decontaminating apparatus filter, Titanium dioxide-based, in the Anatase main form, photocatalytic products have been applied by spray-coating in an amount equal to about 100 g/m². Coating of the filter present in the antimicrobial section has been carried out with the Bactercline Multiuso bactericidal product in an amount equal to ca 60 g/m² of product.

The air contained in the Smog Chamber first compartment has been contaminated with the aid of a nebuliser of the New Triflux 400 type, NUCLEOFARMA, the nozzle of which has been inserted in the hole, which is present in the Smog Chamber first compartment. The nebulization rate, which is dictated by the instrumental characteristics of the nebulizer, is of 0.22 ml/minute, and the dimensions of the nebulised particles, composed of aqueous suspensions of bacteria, have an average diameter of ca. 2.6 μm.

The forced movement of air was carried out by the fan that was contained in the decontaminating equipment. The turning on of the fan causes the passage of air through the filters, from the first to the second compartments of the Smog Chamber. Part of the bacteria passing from the first to the second compartment of the Smog Chamber are hold by the filter.

The ability of filters treated with titanium dioxide-based products to perform a bactericidal action under UV illumination has been initially assessed. Furthermore, the bactericidal action of filters treated with Bactercline Multiuso has been assessed in trials performed in the absence of UV illumination.

### Experimental Methods

6 mL of an E. coli suspension diluted at concentrations ranging between 8.0×10⁵−4.0×10⁷ cfu/mL (working culture) has been placed in the nebulizer ampoule. The filtering device and the nebuliser have been turned on and kept operating for 15 minutes, in the case of the trials with photocatalytic products, and for 5 minutes in the trials with Bactercline Multiuso. The nebuliser vaporizes about 1 mL suspension in a period of time of 5 minutes.

The Smog Chamber has been contaminated each time with high amounts of bacteria, to get a neat indication of the efficacy of the photocatalytic products and the Bactercline Multiuso product. In the presence of polyester filters, ca 50% of these cells was blocked in a single pass on the filter. A comparable efficiency has been found by using glass wool filters.

At the end of each experiment, the Smog Chamber has been sterilized by means of a 70% ethanol solution nebulised within the Smog Chamber for a period of time of one hour, and then rinsed with sterile water.

At the end of the nebulisation, the decontaminating device has been kept turned on for ahs in order to assess the microbicidal activity of the irradiated photocatalytic filters which were present in the Photocatalytic section, and for a period of time equal to minutes, in order to assess the activity of the filters present in the antimicrobial section. Once the activation times of the device were elapsed, the Smog Chamber has been opened, and the filters have been quickly removed. These have been cut in squared specimens of 2 cm side, placed in Petri dishes, and covered with 15 mL liquid agarized culture medium, kept at a temperature of 50° C. The Petri dishes have been kept under slight stirring for 1 minute, in order to promote the diffusion throughout the plate of the residual bacteria on the filter specimen, and the medium was left to solidify at room temperature. Finally, the Petri dishes have been placed into an incubation cell at 37° C. for 24 hours. At the end of this period, a counting of the colonies for each plate was performed. Within the scope of each pair of experiments, with the lamp being turned off and on, the number of bacterial colonies detected on the filters after the relative times of turning on of the device has been compared, in the absence and in the presence of UVA light. In this manner, it has been possible to determine the mortality of the bacteria due to the presence of UVA light and to the treatment with photocatalytic products. Table 1 reports the results of the tests that were carried out in the Smog Chamber, with the filters non-treated and treated with the photocatalytic products, under off and on UVA lamp conditions.

TABLE 1
Assessment of the mortality of the micro-organisms (E. coli) hold
by the polyester filters, non-treated and treated with the photocatalytic
products, in the absence and the presence of UVA illumination.

| | Control | Control | Treated | Treated |
|---|---|---|---|---|
| | filters | filters | filters | filters |
| | UV OFF | UV ON | UV OFF | UV ON |
| Cfu/plate3 h | 2.07 × 10³ | 1.16 × 10³ | 3.29 × 10³ | 8.90 × 10¹ |
| Reduction % | / | 54% | / | 98% |
| in 3 h | | | | |

The results reported in Table 1 represent the average of trials repeated under similar conditions. From a comparison of the data of the first two columns of Table 1, it is possible to deduce that about 50% of the mortality observed for E. coli is to be attributed to the UV irradiation apparatus included in the decontaminating device. However, it is interesting to note that in the filters treated with the photocatalytic products under irradiation condition, the mortality of E. coli is almost doubled, in a reproducible manner, reaching the average value of 98% after 3 h ventilation.

In Table 2, the results are reported which were observed on the Bactercline Multiuso-treated filters in the absence of UVA irradiation.

TABLE 2
Assessment of the mortality of the micro-organisms
(E. coli) hold by the polyester filters, non-treated
and treated with Bactercline Multiuso, in the absence
of UVA illumination, after 15 minutes of ventilation.

| | | Bactercline |
|---|---|---|
| | Control | Multiuso- |
| | filters | treated filters |
| | UV OFF | UV OFF |
| Cfu/plate 15′ | >5.00 × 10³ | 0 |
| Reduction % | / | 100% |
| in 15 min. | | |

The results reported in Table 2 also represent the average of 5 trials repeated under similar conditions. As it shall be noted in the first column, after 15 minutes from nebulization of the bacteria, the residual number of the micro-organisms which are present on the non-treated filters is above 5.0×10³ per plate in average. Instead, the second column shows that, after the nebulization of an equivalent amount of bacteria, colonies do not develop on the Bactecline Multiuso-treated filters, indicating the complete mortality of the microbial species which contacted such filters.

A distinct series of trials was to verify, for the Bactercline Multiuso product-treated filters, the presence of a wide-spectrum antimicrobial activity by using mixtures of the following microorganisms:

| | |
|---|---|
| Pseudomonas aeruginosa | ATCC 15442 |
| Staphylococcus aureus | ATCC 6538 |
| Escherichia coli | ATCC 10536 |
| Enterococcus hirae | ATCC 10541 |
| Candida albicans | ATCC 10231 |

Such micro-organisms have been purchased from the companies Diagnostic International Distribution SpA and VWR International Srl.

The bacterial strains have been kept frozen in culture broth and 50% glycerol (v/v); before their use, they have been transplanted on TSA slant and kept in a refrigerator at 4° C.±2° C.

Candida albicans has been kept frozen in culture broth and 50% glycerol (v/v); before its use, it has been transplanted on Malt Extract Agar slant and kept in a refrigerator at 4° C.±2° C.

### Culture Media

Tryptone Soya Agar (TSA) for the bacterial strains, and Malt Estract Agar (MEA) for Candida albicans.

In this series of trials, known amounts of mixtures of bacteria (Escherichia coli, Staphyloccoccus aureus, Pseudomonas aeruginosa, Enterococcus hirae) and fungi (Candida Albicans) have been contacted with polyester and glass wool filters specimens, treated with Bactercline Multiuso. Then, the antimicrobial power of the treated filters has been assessed, after a contact time of 15 minutes with the microbial mixture, comparing the results with those of similar control trials carried out with non-treated filters.

The results obtained indicated for the Bactercline Multiuso product-treated filters a neat reduction of the micro-organisms, exceeding four logarithms, compared to the control filters.

### Assessment of the Overall Efficiency of the Two—Photocatalytic and Antimicrobial—Sections

The overall decontaminating efficiency of the inventive equipment has been assessed by comparing the bacterial load which was present in the Smog Chamber second compartment after a filtration period of 15 minutes, with the bacterial load being detected under the same conditions in the absence of filters in the filtering device (control trials).

The air sampler of the “SAS100” type has been inserted, during sampling, in a special opening which was present on the second compartment side.

### Procedures and Results

3 mL of an E. coli suspension diluted to concentrations ranging between 1.5×10⁴−2.0×10⁵ cfu/mL (working culture) has been put in the nebulizer ampoule.

Before contamination, the sampling of the air in the Smog Chamber second compartment (indicated as sampling at Time 0) has been performed in order to verify the absence of aero-dispersed micro-organisms. Once the sampling at Time 0 was completed, the filtering device and the nebuliser have been turned on and kept operating for 15 minutes, the period of time in which the amount of 1 mL working culture is vaporized.

Typically, working cultures with concentrations of the order of 5.0×10⁴ cfu/mL have been used in order to contaminate the Smog Chamber first compartment with an overall number of about 50,000 bacterial cells.

In trials which were performed in the absence of filters, it has been noted that in a period of time of 5 minutes, the number of colonies that were transported by the non-filtered ventilation system corresponded to 5-6% of the bacterial cells. In the presence of filters, about 50% of these cells were blocked in a single passage on the filter.

The air filtration from the first to the second compartment of the Smog Chamber was activated concomitantly to the nebulisation of the bacteria. At the end of the nebulisation, the filtering device was turned off, and the sampling of the air in the second compartment was performed. At the end of the sampling, the Plate Contact Agar (PCA) plates, which were used with the SAS100 sampler, were put in an incubation cell at 37° C. for 24 hours, then the number of colony-forming units per plate (cfu/plate) was assessed. At the end of each experiment, the Smog Chamber was sterilized by means of a 70% ethanol solution nebulised within SC for a period of time of one hour, and then rinsed with sterile water. Table 3 reports the results of the performed tests.

TABLE 3
Assessment of the activity of the decontaminating device.

| | Average cfu/plate detected in the | | |
|---|---|---|---|
| | second compartment compared to the | | |
| | corresponding controls (between brackets) | | |
| | Sampling | | |
| Type | Time 0 | Sampling | Reduction |
| of filter | 50 litres | 20 liters | % |
| Polyester | 0 | 178 (480) | 63% |
| Wool Glass | 0 | 215 (530) | 59% |
| Treated | | | |

The values in the table represent the average of 5 different trials, and have an undetermination of 10%.

From the data reported in Table 3, the efficacy in reducing in a short period of time (15′) the microbial load passing therethrough of the device containing the two-Photocatalytic and Antimicrobial-sections will be apparent.

### Efficiency of the Decontaminating Apparatus in Reducing Nitrogen Oxides, NOx

The efficiency of the apparatus in decontaminating chemical pollutant species was assessed by considering mixtures of nitrogen oxides with a high concentration.

The measurements of the concentration of the initial NOx (in the range from 0.6 to 0.7 ppm) and at different irradiation times were performed by following a chemiluminescence-based analytical method, illustrated in UNI 10878 standard.

For the measurements of NOₓ reduction, the gas phase concentration as a function of time has been monitored, under conditions of recirculation of the gas through the decontaminating equipment of the invention, with the Photocatalytic section being illuminated and not illuminated.

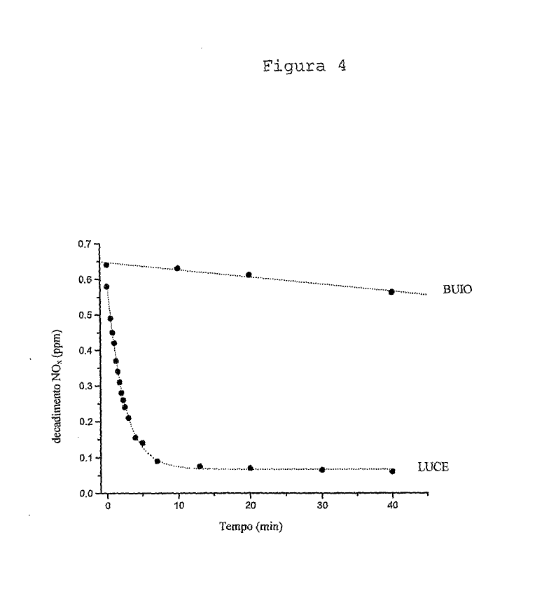

The results reported in FIG. 4 indicate that, in a period of time of the order of 10 minutes, the apparatus is capable of reducing initial concentrations of nitrogen oxides of 0.65 ppm.

Therefore, it shall be apparent that the decontaminating equipment of the invention achieves the intended objects, obtaining in few minutes an almost complete elimination both of the bacterial and viral load of air, and of pollutants, such as NOx, and odours. What is also significant is that, with the equipment of the invention, the air sterilization and clean up are jointly and simultaneously obtained, while the two treatments occur in different times with the devices of the prior art.

Furthermore, it has been observed that the prearrangement in a sequence of the anti-bacterial/anti-viral section and the photocatalytic section allows optimizing the treatment and extending the useful life of the filters. Without being bound by any theory, in fact, it can be hypothesized that the photocatalytic treatment, in the second section, of air which has already been sanitised by the antibacterial treatment performed by the nanocrystalline materials of formula (I), is quicker and more efficient, thanks to the fact that all the reactive sites of the photocatalytic material are available to catalyze the degradation chemical reactions of the pollutant species.

Therefore, a further object of the invention is a method for the treatment of air, comprising i) an elimination or reduction step of the bacterial and/or viral load of said air by means of the passage of said air in contact with a material with antibacterial and antiviral activity, and ii) an elimination or reduction step of the pollutants and/or odours from said air by means of the passage of said air in contact with a material with photocatalytic activity.

It shall be apparent that only some particular embodiments of the present invention have been described, to which those skilled in the art will be able to make all those modifications that are necessary to the adaptation thereof to particular applications, without anyway departing from the protection scope of the present invention.

For example, it will be possible to replace the antibacterial materials of formula (I) with other compounds or materials that are capable of serving the same function, such as, for example, polymers charged with antibiotic or anyway sterilizing substances.

1. A decontaminating equipment (1) for the treatment of air, comprising a shell (2) which is divided into a first and a second compartments (3, 4), which are arranged in a contiguous position in any sequence order, in the second of said compartments (3, 4) suction means (6) being arranged, in which one of said first and second compartments (3, 4) is for the antibacterial/antiviral treatment of air, and one of said first and second compartments (3, 4) is for the photocatalytic treatment of air, and comprises UV illumination means (9), said first and second compartments (3, 4) comprising a material with antibacterial and antiviral activity, and a material with photocatalytic activity, respectively.

2. The equipment according to claim 1, wherein said material with antibacterial and antiviral activity comprises nanocrystalline compounds of formula (I):
AOₓ-(L-Meⁿ⁺)ᵢ (I)
where
AOₓ represents a metal or metalloid oxide, with x=1 or 2;
Meⁿ⁺ is a metal ion with antibacterial activity selected from Ag⁺ and Cu⁺⁺;
L is a bifunctional molecule, organic or organometallic, capable of concomitantly binding both the metal or metalloid oxide and the metal ion Meⁿ⁺; and
i represents the number of L-Meⁿ⁺ groups linked to an AOₓ nanoparticle, in which i ranges between 10² and 10⁶.

3. The equipment according to claim 2, wherein said AOₓ metal or metalloid oxides are selected from colloidal silica, titanium dioxide, zirconium dioxide, tin dioxide, and zinc oxide, and in which L is an organometallic complex comprising an organic ligand, coordinated at a metallic centre, bearing boronic, B(OH)₂, phosphonic, PO₃H₂ or carboxyl, COOH, functionalities, and groups, coordinated at the metallic centre, capable of bonding metal ions with antibacterial activity.

4. The equipment according to claim 3, wherein said groups capable of bonding metal ions with antibacterial activity are selected from Cr⁻, Br⁻, I⁻, CNS⁻, NH₂, CN⁻, and NCS⁻.

5. The equipment according to claim 3, wherein said organic ligand coordinated at the metallic centre is a dipyridyl and/or terpyridyl ligand functionalized with carboxyl COOH, boronic B(OH)₂ or phosphonic PO₃H₂ groups, or in which said dipyridylic and/or terpyridylic groups are substituted with carboxyl groups, preferably in the para position with respect to the pyridine nitrogen or, in the case where more than one dipyridyl and/or terpyridyl group is present in said organometallic complex L, one of said groups can optionally be unsubstituted.

6. The equipment according to claim 2, wherein said metal to which said organic ligands and said groups capable of bonding metal ions with antibacterial activity are coordinated, is a metal of the first, second, or third row of transition in the periodic table of the elements which gives rise to stable bifunctional molecules, preferably selected from Cr, Mn, Fe, Co, Ni, Cu, Zn, Ru, Rh, Pd, Re, Os, Ir, Pt.

7. The equipment according to claim 2, said ligands L being selected from [(H₃Tcterpy)M(CN)₃]TBA, [(H₃Tcterpy)M(NCS)₃]TBA, [M(H₃tcterpy)(bpy)NCS]TBA, and [M(H₂dcb)₂(NCS)₂, where H₃Tcterpy=4,4′,4″-tricarboxy terpyridyl, TBA=tetrabutylammonium cation, bpy=2,2′-dipyridyl, and H₂dcb=4,4′-dicarboxy-2,2′-dipyridyl acid.

8. The equipment according to claim 2, wherein L is an organic molecule containing carboxyl COOH, phosphonic, PO₃H₂, and boronic, B(OH)₂, functionalities, capable of promoting the adsorption onto the surface of the AOₓ oxide, and groups N, NH₂, CN⁻, NCS⁻, CNS⁻, or SH, capable of bonding metal ions with antibacterial activity, said ligand L being selected from:
nitrogen-containing heterocycle with 6-18 members, substituted with one or more substituents selected from carboxyl COOH, boronic group B(OH)₂, phosphonic group PO₃H₂, mercaptan SH, hydroxyl OH;
C6-C18 aryl, preferably selected from phenyl, naphthyl, diphenyl, substituted with one or more substituents selected from carboxyl COOH, boronic group B(OH)₂, phosphonic group PO₃H₂, mercaptan SH, hydroxyl OH;
C2-C18 mono- or di-carboxylic acid, substituted with one or more mercaptan SH and/or hydroxyl OH groups.

9. The equipment according to claim 1, wherein said material with antibacterial and antiviral activity further comprises a cationic surfactant selected from an alkylammonium salt, preferably selected from quaternary ammonium compounds, C12-C14 benzyl, C1-alkylammonium chlorides, benzalkonium chloride, or chlorhexidine digluconate.

10. The equipment according to claim 1, wherein said material with photocatalytic activity is a nanocrystalline material comprising a titanium dioxide layer, preferably in the form of anatase and/or modified peroxytitanic acid.

11. The equipment according to claim 10, wherein said photocatalytic material comprises two or more titanium dioxide layers, preferably in the form of rutile, sandwiched between the treated surface and said first photocatalytic titanium dioxide layer.

12. The equipment according to claim 11, wherein said photocatalytic material comprises one or more further titanium dioxide photocatalytic layers in the form of peroxytitanic acid or other compounds with a strong adhesion power and non-oxidizable, sandwiched between the treated surface and said first photocatalytic titanium dioxide layer.

13. The equipment according to claim 12, wherein said photocatalytic material further comprises titanium dioxide in the Brookite form, and/or stabilizing surfactants.

14. The equipment according to claim 13, wherein said photocatalytic material further comprises at least one component selected from sodium hydroxide (NaOH), lithium oxide (Li₂O), sodium sulfite heptahydrate (Na₂S₂O₃.7H₂O), sodium thiosulphate pentahydrate (Na₂SO₃.5H₂O), and/or silica (SiO₂).

15. The equipment according to claim 1, wherein said material with antibacterial and antiviral activity and said material with photocatalytic activity are arranged on filters, said filters being made of a filtering material selected from:
ceramic material, preferably cordierite;
polymer fibre, preferably synthetic fibre of foamed polyester, impregnated of activated carbons;
polymer fibre, of the type polyester, thermoset polyester, polyurethane, also foamed, in cloth form, also rotative and/or in cup and/or paper form, preferably also impregnated with activated carbons, or entirely filled with activated carbon, or mixed, or impregnated with Zeolite in pellets;
glass fibre with filtering septum in paper of glass microfibres in small plies or deep plies, also with corrugated aluminium separators;
polypropylene (PP), modified polyphenyleneoxide (PPO), polycarbonate (PC), or polystyrene (PS), or in sinterised foamed polystyrene (EPS) composed of a reduced-weight closed-cell rigid foamed material, or mixed.

16. The equipment according to claim 15, wherein also the inner surface of one or more walls of the compartment (4) for the photocatalytic treatment of air is coated with said photocatalytic material.

17. A method for the treatment of air, comprising i) an elimination or reduction step of the bacterial and/or viral load of said air by means of the passage of said air in contact with a material with antibacterial and antiviral activity, and ii) an elimination or reduction step of the pollutants and/or odours from said air by means of the passage of said air in contact with a material with photocatalytic activity.

18. The method according to claim 17, wherein said material with antibacterial and antiviral activity comprises nanocrystalline compounds of formula (I):
AOₓ-(L-Meⁿ⁺)ᵢ (I)
where
AOₓ represents a metal or metalloid oxide, with x=1 or 2;
Meⁿ⁺ is a metal ion with antibacterial activity selected from Ag⁺ and Cu⁺⁺;
L is a bifunctional molecule, organic or organometallic, capable of concomitantly binding both the metal or metalloid oxide and the metal ion Meⁿ⁺; and
i represents the number of L-Meⁿ⁺ groups linked to an AOₓ nanoparticle, in which i ranges between 10² and 10⁶.
